(19) United States
(12) Patent Application Publication
(10) Pub. No.: US 2019/0015248 A1
(43) Pub. Date: Jan. 17, 2019

(54) Post-Breast Surgery Thermal Therapy Apparatus

(71) Applicant: Melissa Russell, Lisle, IL (US)

(72) Inventor: Melissa Russell, Lisle, IL (US)

(21) Appl. No.: 16/032,703

(22) Filed: Jul. 11, 2018

Related U.S. Application Data
(60) Provisional application No. 62/531,670, filed on Jul. 12, 2017.

(51) Int. Cl. A61F 7/02; A61F 7/10

(52) U.S. Cl. CPC A61F 7/02; A61F 7/10; A61F 2007/0019; A61F 2007/0234; A61F 2007/023

(57) ABSTRACT

The present disclosure provides a post-breast surgery thermal therapy apparatus including a vest including at least two compartments, wherein each compartment can accommodate at least one thermal therapy source (e.g., ice pack, frozen veggies, heat packs, etc.). The apparatus can include a neck strap and waist strap to secure the vest body to the chest area of a user.

### CROSS-REFERENCE TO RELATED APPLICATIONS

This application incorporates by reference and claims the benefit of priority to U.S. Provisional Application 62/531,670 filed on Jul. 12, 2017.

### BACKGROUND OF THE INVENTION

Breast surgery comes in many forms. Whether it be breast enhancement, augmentation, reduction, removal, reconstruction, or any other surgical procedure, there can be a great deal of both physical and emotional pain associated with such procedures and the healing process which follows.

Typically, post-surgery, someone having undergone such a procedure is given pain medication and told to apply ice to the area(s) which were operated upon. Given that the overuse of pain medication is dangerous, the use of ice packs and/or frozen vegetables to relieve breast surgery pain is very common. A typical recommendation is to ice breasts for 20 minutes, every hour, for 2-3 days post-surgery and then as needed beyond the first 3 days. This requirement means ice, ice packs, bags of frozen vegetables, etc. must be held in place upon the breast(s) virtually all day for around half a week.

Disk-shaped ice packs can be placed into a brassiere for application of thermal therapy to breasts, but these too are ineffective for those who have recently undergone breast surgery as such patients are unable to wear a bra for several weeks post-surgery and the amount of ice packs or other cold objects a bra can comfortably hold is very limited.

Currently, there is no apparatus that enables thermal therapy (e.g., ice packs, frozen foods, or even hot packs if needed) to be applied effectively to the breasts in a comfortable, clandestine, or functional manner. Patients are left bedridden or with limited mobility post-surgery, as laying still on one's back is the only reliable way to keep a cold source in place upon the breast(s). The use of the hands to hold ice packs, etc. in place limits mobility and the use body wraps, ACE bandages, etc. places painful pressure upon the operated areas.

Because there is currently no product which allows for proper icing without being immobile, patients must either commit to laying/sitting for the initial recovery or elect to not ice for the recommended amount of time which leads to pain and less than optimal surgery results. It is the recommendation of the medical field to be up and moving as much as possible after having undergone anesthesia; however, this is impossible when the patient must be immobile for so long in order to reduce pain and swelling.

Accordingly, there is a need for an apparatus that enables the post-surgery application of thermal therapy to a patient's chest such that a patient is not immobilized or hand-bound during application.

### BRIEF SUMMARY OF THE INVENTION

To meet the needs described above and others, the present disclosure describes an apparatus that enables the post-surgery application of thermal therapy to breasts in a comfortable, clandestine, and functional manner.

In an example, the post-breast surgery thermal therapy apparatus includes at least two fabric pouches wherein each pouch can accommodate at least one thermal therapy source (e.g., ice pack, frozen veggies, heat packs, etc.), a neck strap, and back strap. The pouches are held together via the straps. In an example, the straps can be secured to the user by a center fastener (e.g., buckle, clip, tie, Velcro™, etc.) which enables the apparatus to be securely fastened and unfastened. The straps can be adjustable to allow for a customized fit for each user.

In an example, the post-breast surgery thermal therapy apparatus includes a neck strap, vest portion, two wing portions, and at least one waist straps. The neck strap can be made of ribbed cording and adjustable for different neck sizes via a fastener. The vest portion can include at least two compartments (e.g., 3, 4, 5, or more) that can house a thermal source. The compartments are large enough to cover the areas on, above, and/or under the bust. The center portion of the vest portion can include a fold over flap and fastener in order to secure the thermal source (e.g., packs) in place even during movement by the user. Ideally, the back portion of the vest portion can fit flush against the body can be made of a sport mesh material to let the maximum amount of cold come through. The front of the vest portion will be made of a material that does not retain water and is flexible (e.g., slightly stretchy, elastic, etc.). The two wing portions can hold the vest against the body as much as possible, without constricting or putting too much pressure on the chest and/or breasts. The waist straps can include two strips of ribbed cording along the neck that can be sewn to the edge of the wing portions. A user can wrap (e.g., crisscross) the straps in the back and bring them around to the front to tie in a bow, knot, engage a fastener, etc. for a secure fit. In an example, a specialized frozen gel bead packs can be as the thermal source to improve thermal transfer to breasts as well as comfort of the user.

The present apparatus can enable icing or heating of the breasts post-surgery in a manner which enables freedom of movement and a dignified appearance. The present apparatus can enable both via its innovative design which allows for ice and/or heat to be applied to the breast in and hands-free manner, with the whole breast being covered by ice if need be. Additionally, the present invention conceals the thermal source (e.g., frozen vegetables, etc. which are commonly suggested by doctors for icing breasts) enabling a post-surgery user to apply such cold sources in public without drawing attention to themselves.

An advantage of the present apparatus is the adjustable nature of the apparatus. As it is designed specifically to apply thermal therapy to a bust, the adjustable straps enable a customized fit which ensures ice can be applied to the entire breast(s) of each user no matter their shape or size. This is of critical importance and an exceptionally meaningful advancement for users. The effectiveness of the apparatus is greatly enhanced when the thermal therapy is applied to the user's entire breasts. Additionally, the wing portions on the side allow the apparatus to be one size fits all, but still pulled tight against the body ensuring the thermal therapy is applied consistently while the apparatus is in use.

Another advantage of the present apparatus is the ribbed cording that secures the apparatus at the waist. After breast surgery, it is very difficult to move one's arms up or to the side. The ribbed cording at the waist enables a user to simply crisscross the cords behind the lower back and bring them to the front without the need for any other adjustment or painful arm motions.

Yet another advantage of the present application is that the apparatus provides a hands-free application that does not apply pressure to the chest and which does not provide binding in a manner to limit the patient's mobility and functionality. In addition, the wings of the apparatus are designed to prevent intense pressure to the sore chest and instead directs the pressure around the waist of the patient when wearing the apparatus.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a front view of an embodiment of a post-breast surgery thermal therapy apparatus.

FIG. 2 is a diagram illustrating the dimensions of an embodiment of a post-breast surgery thermal therapy apparatus.

FIG. 3 is a front view of an embodiment of a post-breast surgery thermal therapy apparatus in use.

FIG. 4 is a view of the back side of a user wearing an embodiment of a post-breast surgery thermal therapy apparatus.

FIG. 5 is a view of the side of a user wearing an embodiment of a post-breast surgery thermal therapy apparatus.

FIG. 6 is a front view of the compartments of the vest portion of an embodiment of a post-breast surgery thermal therapy apparatus.

FIG. 7 is a front view of a wingless embodiment of a post-breast surgery thermal therapy apparatus.

FIG. 8 is a front view of a two-compartment embodiment of a post-breast surgery thermal therapy apparatus.

FIG. 9 is a front view of a multi-compartment embodiment of a post-breast surgery thermal therapy apparatus.

### DETAILED DESCRIPTION OF THE INVENTION

The present disclosure describes an apparatus that enables the post-surgery application of a thermal therapy to a patient's chest and/or breasts in a comfortable, efficient, conspicuous, and functional manner. The apparatus can be used in post-surgical environments, providing therapy to reduce bruising, swelling, and pain that typically accompanies mastectomies, breast implants, breast reductions, chest surgery, chest injuries, heart surgeries, and/or sore chest and torso muscles. Although the primary example disclosed herein applies the therapy vest to a user's chest, it is contemplated that the apparatus could be used to alternatively, or in addition to, provide a thermal source to various areas of the user's back.

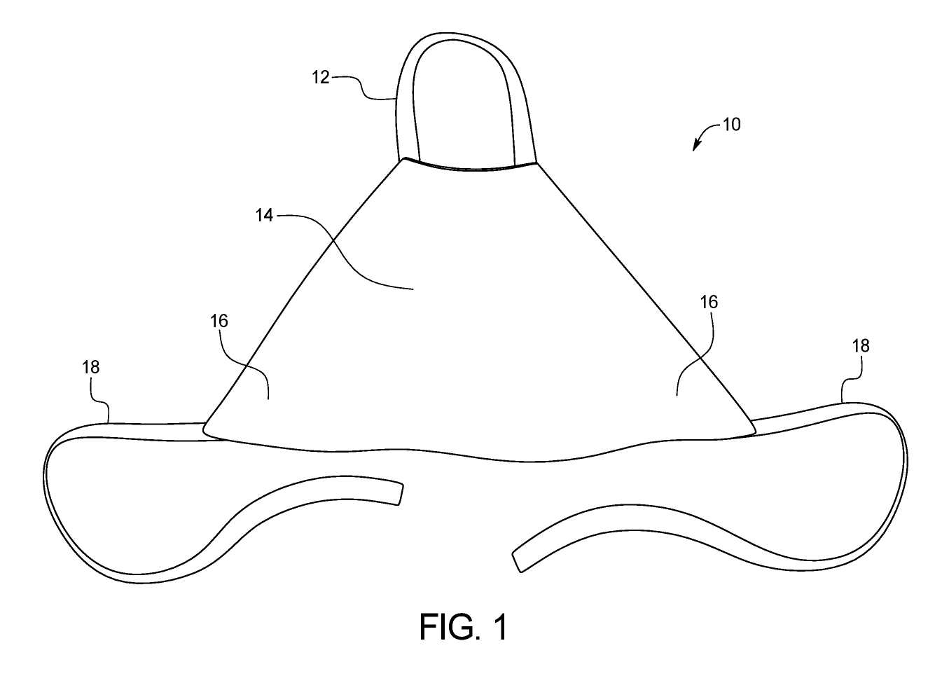

FIG. 1 is an example of a front view of the post-breast surgery thermal therapy apparatus 10. As shown in FIG. 1, the apparatus 10 may feature a neck strap 12, vest portion 14, wing portions 16, and waist straps 18. The neck strap 12 and/or waist strap 18 can be adjustable to improve comfort, fit, and function. The neck strap 12 can be two separate straps that can tie together or come together via a fastener. Similarly, the waist strap 18 can be two separate straps that can tie together or come together via a fastener.

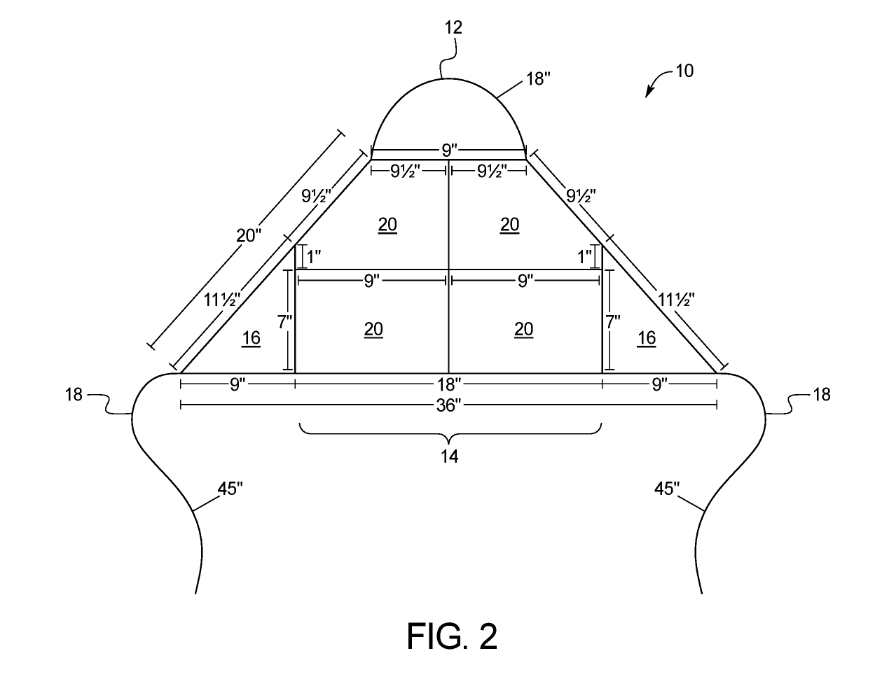

The vest portion can include at least two compartments 20 that can open via a resealable closure, wherein the compartments 20 can house a thermal source 22. As shown in FIG. 2, the apparatus 10 can include four compartments 20. The thermal source 22 can be any suitable thermal source including, but not limited to, ice packs, frozen items, heating packs, heating pads, among others. The resealable closure can include, but not limited to, flaps, snaps, zipper, Velcro™, buttons, hooks, among others.

The vest portion 14 can be composed of any suitable material including, but not limited to, fabric, various synthetic fabric blends, insulation material, among others. The compartments 20 can be made of any suitable material. For example, the compartments 20 can include insulation material to maintain the temperature of the input thermal source. In addition, the compartments 20 can include a water proof lining such that as the thermal source loses the initial temperature, any moisture resulting from thawing, for example, does not cause a problem to the user's clothes.

FIG. 2 also illustrates an example of the dimensions of an example of a post-breast surgery thermal therapy apparatus 10. The neck strap 12 may be 10-22 inches (e.g., 18 inches) in overall length, with a buckle or other fastener enabling it to be shortened as needed for a better fit. The waist straps 18, which wrap around a user, are both 30-55 inches (e.g., 45 inches). The wing portions 16 can be triangular in shape with the bottom edge being 7-10 inches (e.g., 9 inches), the vertical side being 6-10 inches (e.g., 8 inches), and the outer edge (hypotenuse) of the triangular shaped portion being 8-15 inches (e.g., 11.5 inches).

In an example, the vest portion 14 can include four compartments 20. The bottom two compartments can be 9 inches by 7 inches rectangles while the top two compartments can be of an irregular shape best descripted as a rectangle (9 inches by 7 inches) with one corner cut off. The wing portions 16 and vest portion 14 can combine to form an overall shape which can be a frustum (pyramid with its top missing).

The back side of the apparatus may feature breathable mesh to aid in thermal transfer, comfort, and drying of any perspiration by the user. The four compartments 20 which make up the vest portion 14 of the apparatus 10 can be clearly defined by stitching, with the two triangular wing portions also being visible.

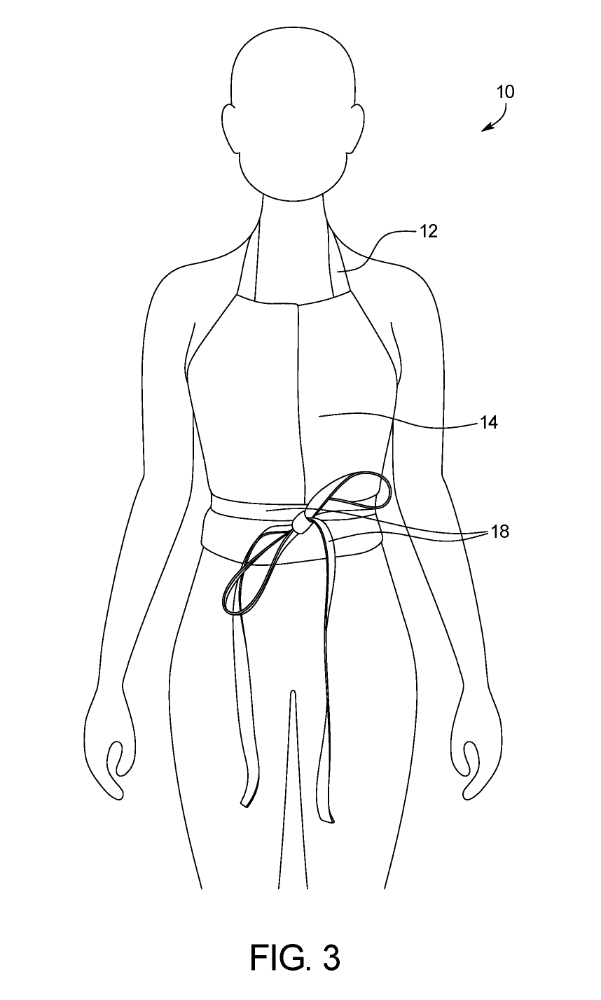

FIG. 3 is a front view of an example of a post-breast surgery thermal therapy apparatus 10 worn on a user. As shown in FIG. 3, a user places the neck strap 12 of the apparatus 10 over their head and wraps the waist straps 18 around themselves, knotting the waist straps 18 in the back or front to provide a secure fit.

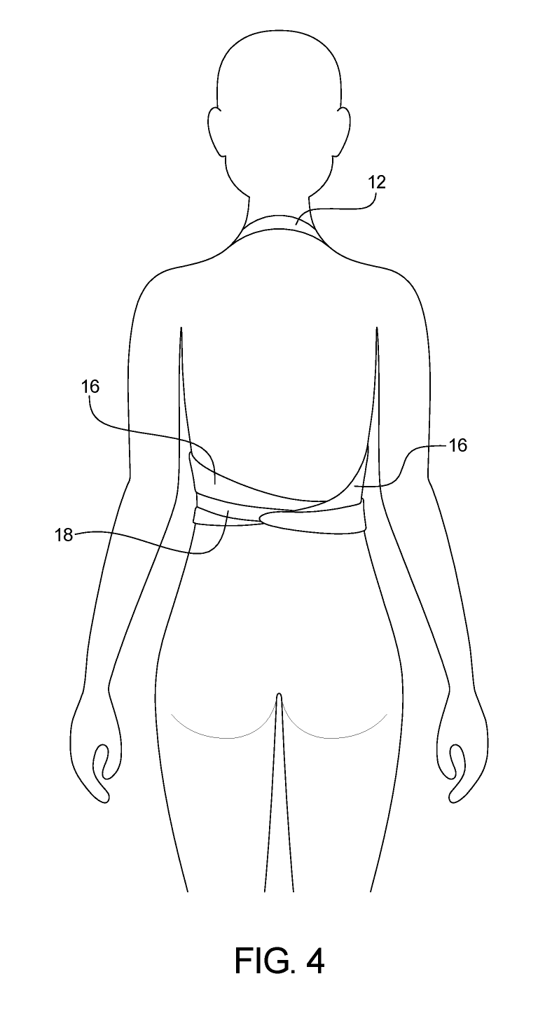

FIG. 4 is a view of the back side of a user wearing the thermal therapy apparatus 10. As shown in FIG. 4, the vest portion is attached to two wing portions 16 of the apparatus 10, which are connected to the waist straps 18, wrap around the side and back of the user. The wings 16 act to provide comfort and secure the apparatus 10 in place and also prevent the waist straps 18 from rubbing or chaffing a user.

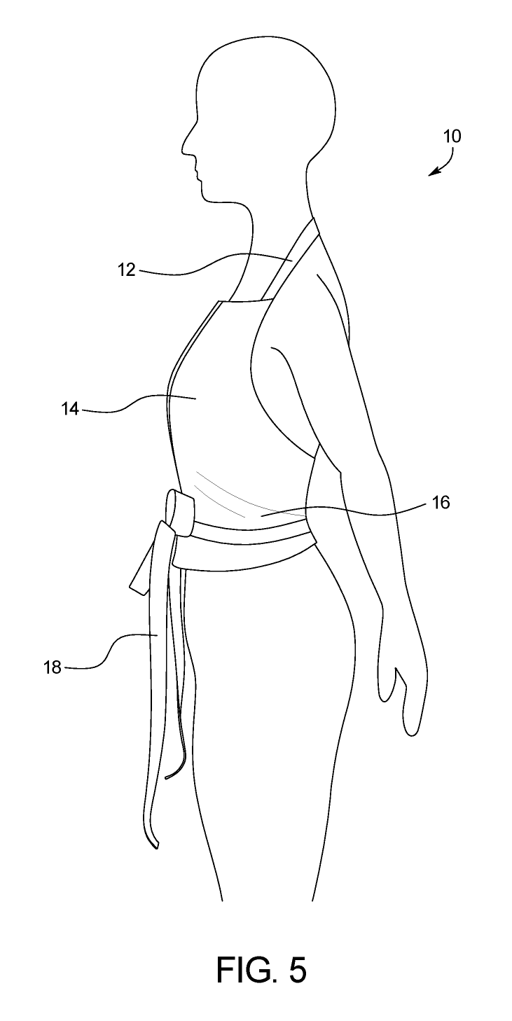

FIG. 5 is a side view of a user wearing the thermal therapy apparatus 10. As shown in FIG. 5, the wing portions 16 of the apparatus 10 act to hold the vest portion 14 (and the ice or other thermal therapy source held therein) tightly in place against the chest/bust of the user. The waist straps 18, connected to these wing portions 16 enable a secure fit to be obtained with minimal arm movement required.

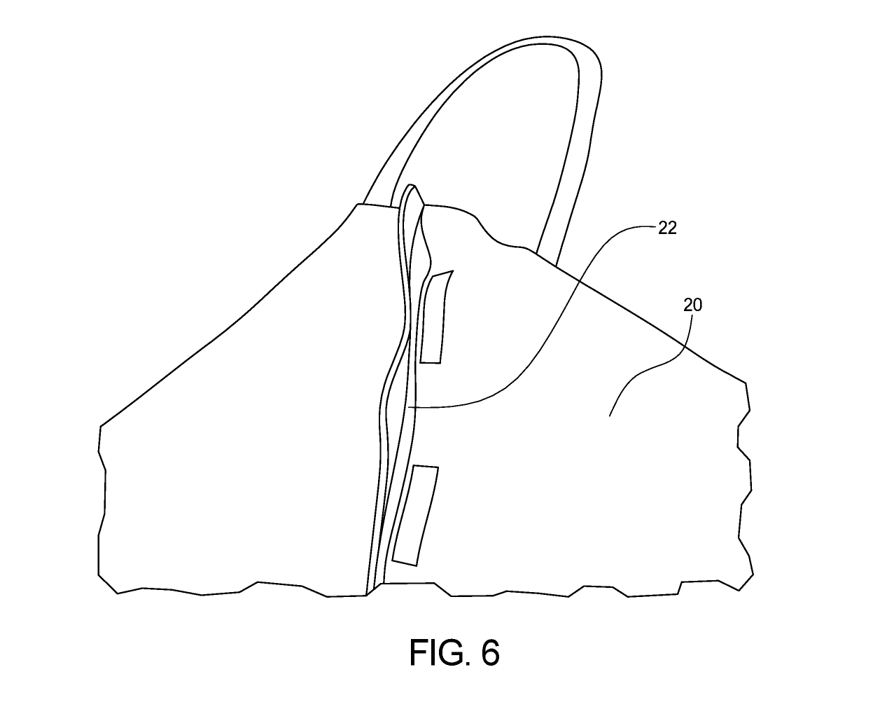

FIG. 6 is a front view of the compartments of the vest portion 14 of the thermal therapy apparatus 10. As shown in FIG. 6, the front of the vest portion 14 of the apparatus features openings for four compartments 20. Of course, it is contemplated the openings to the compartments can be accessed from the back of the vest portion 14. The compartments may each hold a thermal source including, but not limited to, an ice pack, bag of frozen food, frozen gel beads, action activated heat packs, heatable units, etc. and include a resealable opening 22. The openings 22 can include any suitable fastener including, but not limited to, hook, clasp, buttons, snaps, Velcro™, zipper, among others. The opening to the compartment 20 can be accessible from the front of the vest 14 and/or the back of the vest 14.

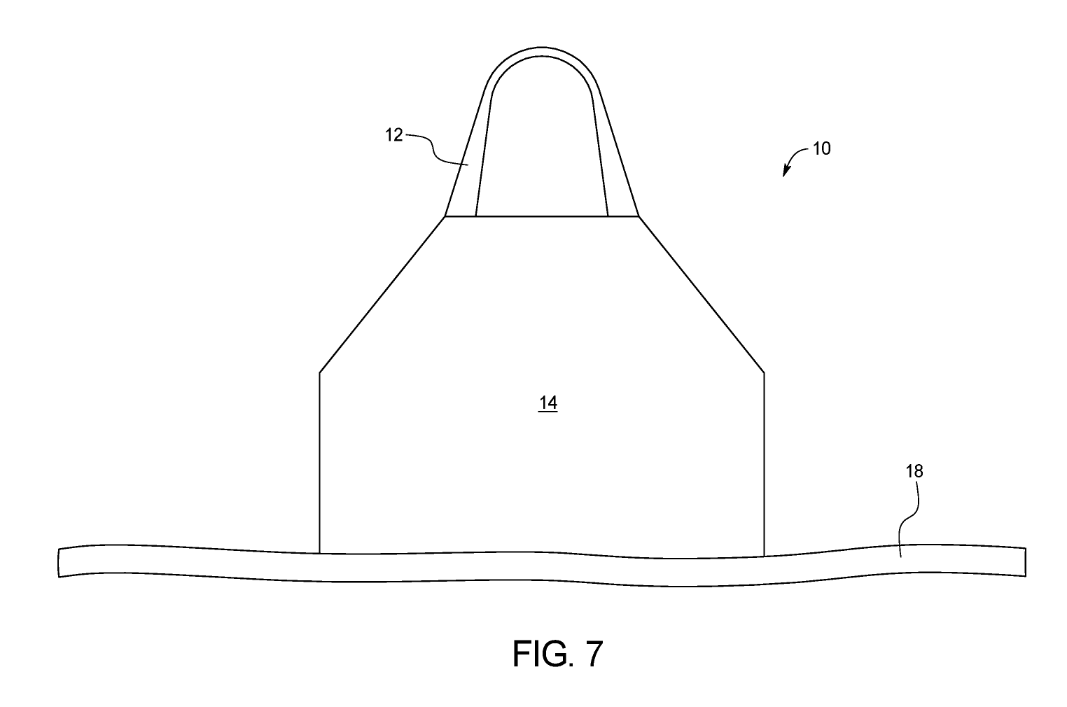

FIG. 7 is a front view of a wingless embodiment of a post-breast surgery thermal therapy apparatus 10. As shown in FIG. 7, the apparatus 10 can also be created without the wings 16 shown in FIGS. 1-6. The rest of the overall dimensions are similar to those of the winged embodiment, with this embodiment potentially being helpful to those who wish for a looser fitting apparatus or one that allows for more airflow. Much like the winged embodiment, the backside of this apparatus embodiment features mesh to enable breathability and moisture wicking. This embodiment also features four resealable compartments 20 which can each accommodate an ice pack, etc.

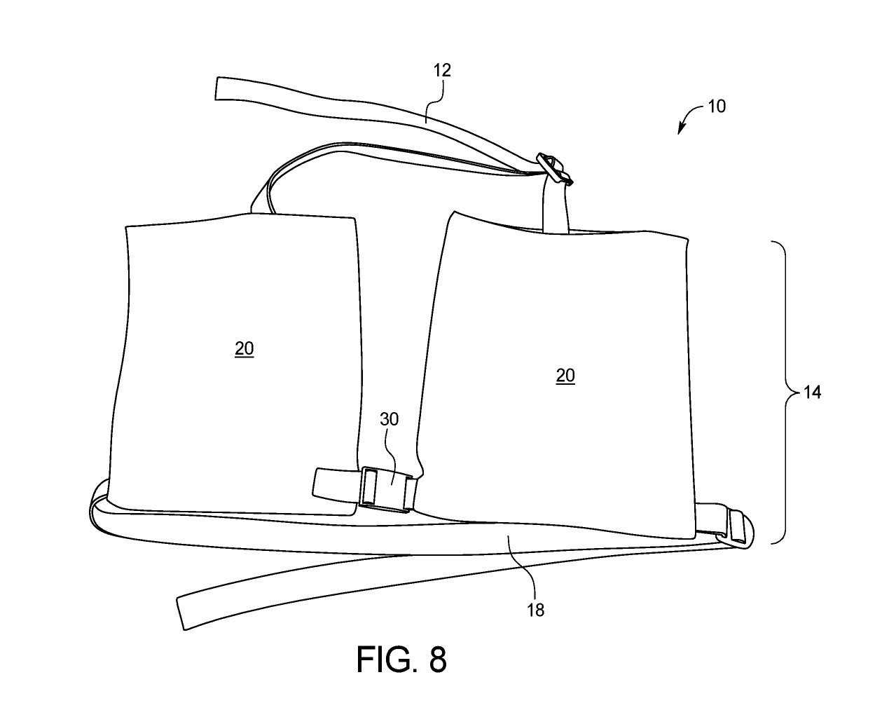

FIG. 8 is a front view of a two-compartment example of a post-breast surgery thermal therapy apparatus 10. As shown in FIG. 8, the apparatus 10 can have two compartment portions 20 which holds the thermal source (e.g., ice packs, etc.). The compartment portions 20 can be connected with the adjustable neck strap 12 and waist strap 18, wherein the compartments 20 (e.g., right chest portion and left chest portion) can be joined via a fastener 30 (e.g., buckle, clasp, hook, etc.). Such configuration can provide a great deal of airflow to the user and puts very minimal pressure on the end user's bust. The configuration in FIG. 8 can accommodate large bags of frozen vegetables as the thermal source, which may be preferable to some users.

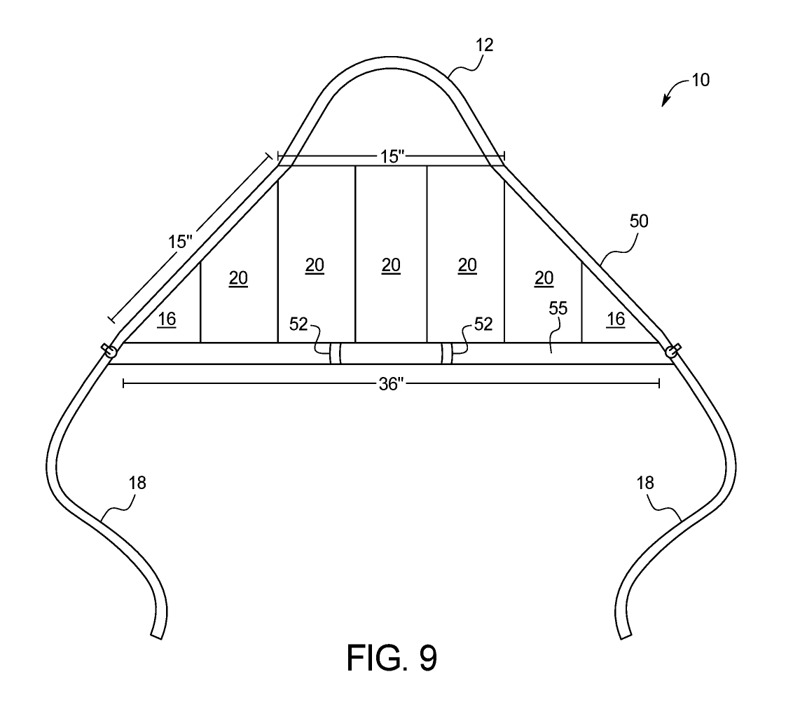

FIG. 9 is a front view of a multi-compartment embodiment of a post-breast surgery thermal therapy apparatus. The apparatus 10 can include a plurality of compartments configured in a variety of manners. In one example, as shown in FIG. 9, the compartments can be parallel to each other extending longitudinally, wherein each compartment 20 includes a top opening for receiving the thermal source. The vest portion 14 can include two to eight compartments, for example, as shown in FIG. 9, five compartments 20. The side wings 16 can attach to the outer most compartments 20. The compartments 20 can include an opening at the top of each compartment for the insertion of the thermal source (e.g., ice pack). Wherein the top portion of the compartment 20 can include at least one snap such that after the thermal source is inserted into the compartment 20, the compartment can be closed via the snap, which pulls the thermal source closer to the chest to allow for cooling of the top of the breast.

In an example, the neck strap 12 and the waist strap 18 can be the same strap. For example, the neck strap 12 can be fed through a strap channel 50 along the outer side perimeter of the vest portion 14, wherein the neck strap 12 can extend out of the strap channel 50 to become the waist strap 18 to secure the apparatus 10 around the waist of a user. The vest portion 14 can include at least one strap loop along the lower perimeter of the vest portion 14, wherein the waist straps 18 can be fed through the strap loops to secure the apparatus 10 to the user. The strap loops enable the vest to curve towards the body to provide cooling to the bottom of the breast. In an example, the waist strap 18 can be attached to a bottom portion 55 of the vest body 14. In an example, the bottom portion 55 can be a bottom border of the vest body 14 adding to further security of the vest on the user.

It should be noted that various changes and modifications to the presently preferred embodiments described herein will be apparent to those skilled in the art. Such changes and modifications may be made without departing from the spirit and scope of the present invention and without diminishing its attendant advantages.

We claim:

1. A vest garment to be secured to a user's torso, wherein the vest garment comprises:
a vest body including at least one compartment, wherein a thermal source can be inserted into the compartment through an opening along at least one edge of the compartment;
a neck strap connected to a top portion of the vest body; and
a waist strap connecting a bottom portion of the vest body, wherein the waist strap can wrap around a waist of the user to secure the vest garment to the user.

2. The vest garment of claim 1, wherein the vest body includes a right chest portion and a left chest portion, wherein each the right chest portion and the left chest portion includes at least one compartment.

3. The vest garment of claim 1, wherein the vest body includes a right chest portion and a left chest portion, wherein each the right chest portion and the left chest portion includes at least two vertically aligned compartments.

4. The vest garment of claim 1, wherein the waist strap includes a fastener for adjusting the size of the waist strap.

5. The vest garment of claim 1, wherein the vest body includes a waist strap channel along the lower edge of the vest body, wherein the waist strap extends through the waist strap channel.

6. The vest garment of claim 1, wherein the vest body includes a triangular wing connected to each side of the vest body, wherein the waist strap connects to each triangular wing.

7. The vest garment of claim 6, wherein the triangular wing includes a compartment for receiving a thermal source.

8. The vest garment of claim 1, wherein the compartments include an inner lining, wherein the inner lining includes a water-proof material.

9. The vest garment of claim 1, wherein the vest body is in the shape of a frustum.

10. The vest garment of claim 1, wherein the vest body includes a right chest portion and a left chest portion, wherein the right chest portion is separate from the left chest portion, wherein the right chest portion is connected to the left chest portion by a fastener.

11. The vest garment of claim 1, further comprising a frozen thermal source in each compartment.

12. The vest garment of claim 1, wherein the neck strap and the waist strap are one continuous strap.

13. The vest garment of claim 12, wherein the vest body includes a strap channel on each side edge of the vest body, wherein the neck strap extends through a top portion of the strap channel and emerges from a bottom portion of the strap channel as the waist strap.

14. The vest garment of claim 1, wherein the vest body includes at least three vertically aligned compartments extending from the top portion of the vest body to the bottom portion of the vest body.
